(19) United States
(12) Patent Application Publication
(10) Pub. No.: US 2019/0040070 A1
(43) Pub. Date: Feb. 7, 2019

(54) PROCESS FOR THE SYNTHESIS OF STABLE AMORPHOUS IBRUTINIB

(71) Applicant: AZAD PHARMACEUTICAL INGREDIENTS AG, Schaffhausen (CH)

(72) Inventors: Thomas MAIER, Stockach (DE); Inna KARAPETYAN, Yerevan (AM); Alvard ARAKELYAN, Yerevan (AM); Tamara MARGARYAN, Kotayk Region (AM); Vardan SARGSYAN, Yerevan (AM); Heghine STEPANYAN, Yerevan (AM); Hermine ABOVYAN, Yerevan (AM); Roman GERBER AESCHBACHER, Frauenfeld (CH); Sven HAFERKAMP, Bochum (DE)

(21) Appl. No.: 16/073,897

(22) PCT Filed: Feb. 8, 2017
(86) PCT No.: PCT/EP2017/052773

§ 371 Date: Jul. 30, 2018

(30) Foreign Application Priority Data
Feb. 9, 2016 (GB) 1602297.2

(51) Int. Cl. C07D 487/04; A61K 9/20

(52) U.S. Cl. CPC C07D 487/04; A61K 9/20

(57) ABSTRACT

Disclosed herein is a new route of synthesis and a new stable amorphous form of ibrutinib. Also disclosed are pharmaceutical compositions, oral dosage forms and the use of the amorphous ibrutinib in the treatment of mantle cell lymphoma or chronic lymphocytic leukemia.

### CROSS-REFERENCE TO RELATED APPLICATIONS

This application is a National Stage of International Application No. PCT/EP2017/052773, filed on Feb. 8, 2017, and published in English as W02017/137446 A1 on Aug. 17, 2017. This application claims the priority to Great Britain Patent Application No. 1602297.2, filed on Feb 9, 2016. The entire disclosures of the above applications are incorporated herein by reference.

### FIELD

Disclosed herein is a new route of synthesis and a new stable amorphous form of ibrutinib. Also disclosed are pharmaceutical compositions, oral dosage forms and the use of the amorphous ibrutinib in the treatment of mantle cell lymphoma and chronic lymphocytic leukemia.

### BACKGROUND

This section provides background information related to the present disclosure which is not necessarily prior art.

Ibrutinib (1-[(3R)-3-[4-Amino-3-(4-phenoxyphenyl)pyrazolo[3,4-d]pyrimidin-1-yl] piperidin-1-yl]prop-2-en-1-one) is a pharmaceutically active drug and belongs to the group of kinase inhibitors, which inter alia are used in the treatment of mantle cell lymphoma and chronic lymphocytic leukemia. The structure of the molecule is displayed in formula I:

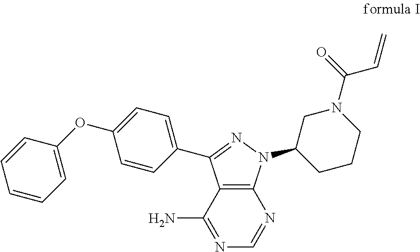 [SMILES: C=CC(=O)N1CCC[C@@H](n2nc(-c3ccc(Oc4ccccc4)cc3)c3c(N)ncnc32)C1]

It is known that ibrutinib is an inhibitor of Bruton's tyrosine kinase (BTK), which is a member of the Tec family of non-receptor tyrosine kinases. This enzyme is an important signaling enzyme expressed in all hematopoietic cell types except T-lymphocytes and natural killer cells. BTK plays a crucial role in the B-cell signaling pathway linking cell surface B-cell receptor stimulation to downstream intracellular responses, especially influencing development, activation, signaling and survival of B-cells. Furthermore, the kinase is also a factor in other hematopoietic cell signaling pathways, for instance Toll like receptor and cytokine receptor mediated TNF-α production in macrophages, IgE receptor signaling in Mast cells, inhibition of Fas/APO-1 apoptotic signaling in B-lineage lymphoid cells and collagen-stimulated platelet aggregation.

Several documents can be found in the literature describing the synthesis of various ibrutinib modifications. WO 2015/081180 A1 for instance disclose the crystalline Form I of ibrutinib, processes for its preparation, pharmaceutical compositions comprising the Form, and use of form I of ibrutinib for treating or delaying diseases or disorders related to activity of BTK proteins. The form was characterized by X-ray powder diffraction, differential scanning calorimetry and other techniques.

A further patent document WO 2013/184572 A1 discloses another crystalline form, form A, of a BTK inhibitor, including solvates and pharmaceutically acceptable salts thereof. Also disclosed are pharmaceutical compositions that include the BTK inhibitor, as well as methods of using the BTK inhibitor, alone or in combination with other therapeutic agents, for the treatment of autoimmune diseases or conditions, heteroimmune diseases or conditions, cancer, including lymphoma, and inflammatory diseases or conditions.

Nevertheless, despite the existing forms of ibrutinib, there is still the need for further routes of synthesis and new modifications of ibrutinib, exhibiting excellent storage stability, low hygroscopicity and good dissolution kinetics.

### SUMMARY

This section provides a general summary of the disclosure, and is not a comprehensive disclosure of its full scope or all of its features.

It has been found that the above mentioned task is inventively fulfilled by a process for the production of amorphous ibrutinib at least comprising the following synthesis steps:

a) reacting compound (1) (3-(4-phenoxyphenyl)-1H-pyrazolo[3,4-d]pyrimidin-4-amine) and an in 3 position functionalized and amino-protected piperidine, wherein the 3 position is functionalized by a leaving group X and the piperidine amino-group is protected by the amino-protecting group Z, to yield compound (2) (1-[(3R)-3-[4-amino-3-(4-phenoxyphenyl)pyrazolo[3,4-d]pyrimidin-1-yl]piperidin-1-yl]Z)

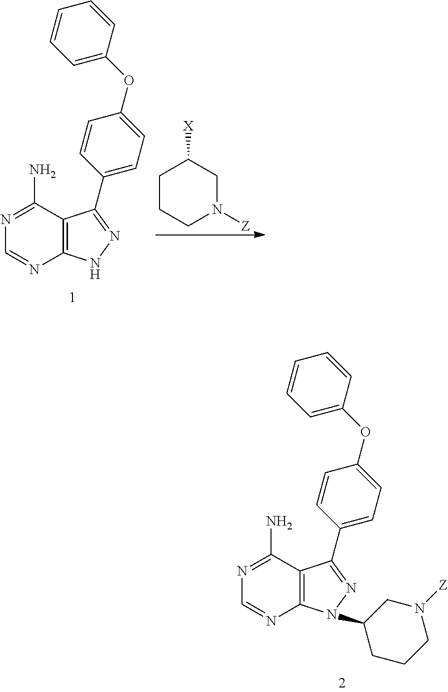

b) deprotection of compound (2) to yield compound (3) (1-[(3R)-3-[4-amino-3-(4-phenoxyphenyl)pyrazolo[3,4-d]pyrimidin-1-yl]piperidin])

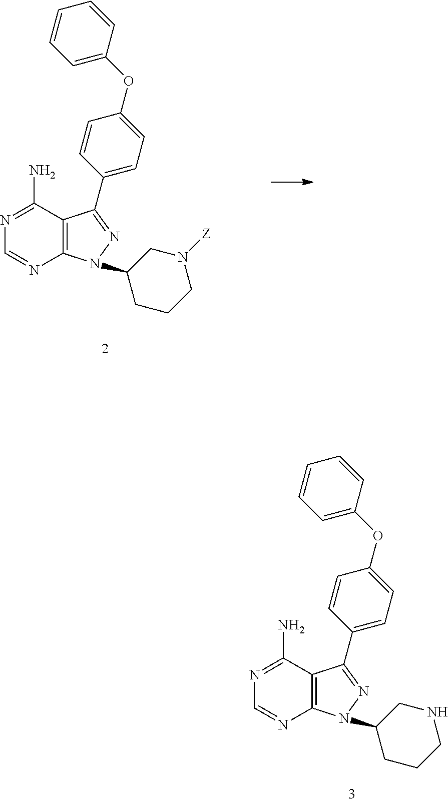

c) reacting compound (3) and acryloyl chloride (2-propenoyl chloride) in an pharmaceutically acceptable solvent to yield compound (4) ibrutinib (1-[(3R)-3-[4-Amino-3-(4-phenoxyphenyl)pyrazolo[3,4-d]pyrimidin-1-yl]piperidin-1-yl]prop-2-en-1-one);

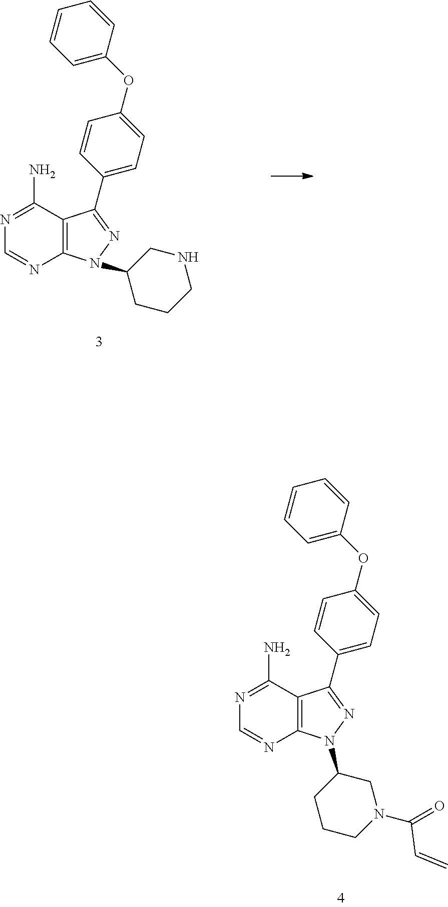 [SMILES: Nc1ncnc2c1c(-c1ccc(Oc3ccccc3)cc1)nn2[C@@H]1CCCNC1 ; C=CC(=O)N1CCC[C@@H](n2nc(-c3ccc(Oc4ccccc4)cc3)c3c(N)ncnc32)C1]

d) filtration of the reaction mixture obtained in step c); and
e) evaporation of the solvent to precipitate essentially amorphous compound (4);
wherein the pharmaceutically acceptable solvent of step c) comprises an electric dipole moment μ/D of ≥2 and ≥8. Surprisingly it has been found that via above described route of synthesis including the physical process steps d) and e) essentially storage stable amorphous ibrutinib is accessible. This finding is especially valid for the complete range of up-scaling and drug processing quantities and surprising, because state of the art processes either directly obtain defined crystalline ibrutinib polymorphs or result in amorphous ibrutinib transforming into defined crystalline polymorphs as a function of time. The latter is especially found in standard processes when larger drug quantities are obtained from solution by precipitation. Without being bound by the theory it is assumed that crystalline ibrutinib represents the thermodynamically most stable drug configuration under standard conditions. Hence, these crystalline polymorphs are usually directly generated in the processes, e.g. by crystallization steps from solution or inherently by equilibrium processes after solvent evaporation during storage. Therefore, even by starting in a standard process at a mostly amorphous form the crystallinity of the API increases with time, resulting in undefined physical or chemical compound properties. The crystallization tendency of standardly prepared ibrutinib might affect for instance the dissolution or hygroscopic properties of the drug and may in consequence also alter bioavailability. This crystallization behavior can inventively be prevented by using the proposed route of synthesis and a precipitation (filtration and evaporation) step, wherein only selected solvents comprising a special electric dipole moment range are utilized. Without being bound by the theory it is assumed that especially the dipole properties of the inventively usable solvents are critical for achieving an amorphous form exhibiting no tendency for crystallization into defined crystalline polymorphs. It is further assumed that the reason for this finding is that the selected range of solvent electric dipole moments is able to shield ibrutinib electrostatic interaction sides between the single molecules, which otherwise result in a symmetric aligning of the drug molecules in the course of solvent evaporation/precipitation. Therefore, a purely random orientation of the molecules is achieved in the solvent-free stage, which is, in addition, less prone to symmetric rearrangements (crystallization) upon storage. This solvent effect is also in part attributable to the filter step, which is potentially able to remove defined polymorphic nucleation centers, which might be generated in the course of the synthesis steps. Furthermore, it was found that the crystallization behavior can also be influenced by the chosen synthesis route. Besides the presence of remaining solvent molecules also the required reaction conditions and the sequence of the generation of possible nucleation/aggregation centers in the molecule seems to influence the overall tendency of the molecule to generate crystalline structures. In consequence, this proposed route of synthesis is able to reduce such risks and therefore also large batches can be processed into a purely amorphous, storage stable forms.

The electric dipole moment of suitable solvent molecules in the above given electric dipole moment range is tabulated, for instance in “CRC Handbook of Chemistry and Physics”, Ed. 2005, pages 9.45 or in Riddick, J. A., Bunger, W. B., and Sakano, T. K., Organic Solvents, Fourth Edition, John Wiley & Sons, New York, 1986. The values of the electric dipole moment of single molecules in the gas phase is used for the here defined dipole moment range. In the case that tabulated values are unavailable the electric dipole moment of the solvent molecules can be assessed by microwave spectroscopy, molecular beam electric resonance or other high-resolution spectroscopic techniques, known to the skilled artisan. The here used dipole moment μ is given in Debye units (D). The conversion factor to transfer the given values into SI units is 1 D=3.33564×10⁻³⁰ Cm.

In the ibrutinib synthesis in step a) a piperidine is used which is functionalized in the 3 position by a leaving group X. Suitable leaving groups in the ortho-position may be selected from —F, —Cl, —Br, —I, —OH, —NH₂, mesylate, triflate, tosylate, diazonium salts, haloalkyl-, alkyl- or aryl sulfonates, phosphates, phosphonic acids, or phosphonic esters and other inorganic esters, wherein the halides and sulfonates are preferred. These leaving groups are technically and commercially feasible, chemically advantageous due to the fact that their use result in a lower amount of impurities and, furthermore, these favor the synthesis or just one enantiomer. Furthermore, educts comprising such leaving groups can be reacted at lower reaction temperatures.

In addition, besides the leaving-group in ortho-position the piperidine in step a) is further amino-protected by the group Z. Suitable groups Z are in general protection groups, which are cleaved under basic, acidic or reductive conditions or alternatively which are cleaved by transition metal catalysis. Those protective groups comprise the advantage that they do not interact during subsequent reactions and can be easily cleaved. Using those protective groups will result in higher chemical yields, cleaner chemical conversions with smaller amounts of side products. Furthermore, the protected derivatives comprise higher solubility, which in turn results in higher volume yields. In principle it is also possible to select the protection group as a function of the further processing conditions. If the further processing is for instance performed in a high pH regime it is helpful to use acid-labile protection groups in order to avoid unwanted cleavage.

In step a) piperidine and compound (1) are reacted to synthesize compound (2). Generally this reaction can be performed in solvents consisting of or comprising substituted or unsubstituted alcohols, alkanes, alkenes or aromatic or heteroaromatic solvents or combinations thereof. The temperature may range from −20° C. to the boiling point of the solvent. The reaction time can be in between 15 h up to 30 h, preferably in between 20 h up to 26 h.

In step b) the deprotection of compound (2) is achieved. Such reaction might be performed in solvents like alcohols or esters in acidic media or under reductive conditions. The deprotection can be performed under mild conditions, for example at ambient temperature, e.g. between 20 and 30° C.

Compound (3) may be used as is in the course of the further synthesis or may optionally be purified by forming a suitable salt with acids, e.g. hydrochloric or hydrobromic acid, acetic acid, tartaric acid, (-) camphor-10-sulphonic acid, 3-bromo-camphor-8-sulfonic acid, mandelic acid, 1-phenylethane sulphonic acid, phenylglycine or mixtures thereof. The crystallization of the suitable salt might lead to a chiral resolution of racemic compound (3) or, alternatively, to an enhancement of the optical purity of compound (3).

The route of synthesis includes the use of a pharmaceutically acceptable solvent in step c). In principle the pharmaceutically acceptable solvent can be selected from the solvents mentioned in the ICH Q3C guidance document (February 2012) in Class 2 or 3 (solvents which should be limited in pharmaceutical products), as long as the solvent comprises the “right” dipole moment according to the disclosure, in order to prevent a ibrutinib crystallization into a defined polymorphic form.

In a further aspect of the inventive process the pharmaceutically acceptable solvent of step c) may comprise an electric dipole moment μ/D of ≥2.5 and ≤5. Such range of electric dipole moments has been proven useful in order to achieve purely amorphous ibrutinib comprising an excellent long-term stability of the amorphous form even at higher temperatures and/or humidities. No crystallization of the amorphous into a defined crystalline form is detectable even at accelerated storage conditions, rendering this amorphous form very suitable for a pharmaceutical processing. Without being bound by the theory it is assumed that solvents comprising the above mentioned range of electric dipole moments are able to sufficiently dissolve possible nucleation centers formed in the course of the synthesis and later on are able to prevent the crystallization into defined polymorphs upon evaporation of the solvent. Such behavior might be attributed to a selective adherence of the solvent molecules to polar drug moieties, favoring a random drug precipitation instead of defined crystal formation.

Within a preferred characteristic of the inventive process the pharmaceutically acceptable solvent of step c) comprises an electric dipole moment μ/D of ≥2.5 and ≤5 and a boiling point ≥75° C. ≤200° C. Besides the “right” electric dipole moment of the solvent molecules also the boiling point of the solvent might influence the precipitation behavior of the ibrutinib. It has been found that solvents comprising a combination of above mentioned electric dipole moments and boiling points are especially suitable for forming amorphous precipitates instead of defined crystalline polymorphs even at large scale batch sizes. Without being bound by the theory this might be attributable to a preferred evaporation speed of the solvent, resulting in a fast precipitation process, wherein the timescale for thermodynamically favorable crystalline re-arrangements is limited, favoring a random orientation of the drug molecules upon solvent removal.

In a further embodiment the solvent in step c) can be selected from the group consisting of MEK, benzonitrile or mixtures thereof. Particularly the precipitation of ibrutinib from MEK or benzonitrile seems suitable to generate a purely amorphous ibrutinib precipitate with a very low tendency for crystalline re-arrangements in the dry or semi-dry state.

In a further characteristic of the process the leaving group X of the piperidine in step a) can be selected from the group consisting of halide, mesylate, triflate, tosylate, benzenesulfonate. These leaving groups X result in a high reaction rate between the piperidine and compound (1) even at mild reaction conditions, thus reducing the risk of the formation of unwanted side-products. Although this reaction is performed at mild conditions high volume yields are obtainable and it is technical feasible to include further purification steps after the chemical reaction.

Within another aspect of the inventive process the amino-protecting group Z of the piperidine in step a) is selected from the group consisting of Boc, Cbz, Tosyl, Mesyl, Triflat, Benzyl, Fmoc, substituted or unsubstituted Acetyl, Benzoyl, Tolyl. Such protecting groups are able to effectively protect the nitrogen function of the compounds (4)-(7) and (8) and can be removed under gentle conditions, not interfering with the following transformation steps. Preferred groups Z may further be selected from the group consisting of Boc, Cbz or Benzyl.

In a further embodiment of the inventive process the deprotection step b) can be performed acid or metal catalyzed. Preferred acids suitable for performing the deprotection in step b) can be selected from the group consisting of sulfonic acids, sulfuric acid or hydrogen halides. Within this group especially hydrogenchloride and methanesulfonic acid are preferred.

In another preferred embodiment the filtration step d) may comprise passing the reaction mixture of step c) through a filter medium comprising pores sizes in the range of ≥0.001 μm and ≤5 μm. This filtering step is able to exclude particles comprising sizes (longest distance within the particle) larger than 5 μm from the precipitation in step d). Due to this size-exclusion step larger nucleation centers are removed from the solvent, which may comprise ordered crystalline structures. Therefore, the precipitation starts without any defined polymorphic crystals favoring the precipitation of the amorphous drug form. In addition to the above given pore sizes it is possible to also use filter comprising pore-sizes of ≥0.01 μm and ≤2 μm, preferably of ≥0.1 μm and ≤1 μm. These pore-size ranges are able to favor the precipitation of purely amorphous and storage stable ibrutinib.

According to a preferred characteristic of the inventive process the filtration step d) can be performed in a temperature range of ≥25° C. and ≤100° C. For the precipitation of purely amorphous ibrutinib it was found that a temperature-range starting from ambient temperatures up to 100° C. is very suitable for the given solvents. Within this temperature range purely amorphous material is precipitated in an acceptable processing time. This effect might be attributed to the combination of the inventively preferred solvents and the evaporation speed resulting from that temperature range. Furthermore, this temperature range is able to assure a consistent electric dipole range for the solvents, due to the fact that the electric dipole moment can be a function of the temperature. Additionally preferred temperatures for the filtration step d) can be in the range of ≥40° C. and ≤80° C. and further preferred ≥45° C. and ≤60° C. These ranges may further support the dissolution of crystalline material without unsuitable alteration of the temperature dependent electric dipole moment. In a preferred embodiment of the disclosure the filtration step d) and the evaporation step e) can be performed in the same temperature-range.

Within an additional aspect of the disclosure the evaporation step e) can be performed at a pressure of ≥900 hPa and ≤1200 hPa. Such pressure range might enhance solvent removal without increasing the risk of the formation of crystalline precipitates at the phase boundary liquid/gas due to an evaporation cooling effect. Thus, fast processing times are achievable.

In a further aspect of the inventive process the evaporation step e) can be started immediately after the filtering step. In the sense of this application this means that after the last solvent of a batch has passed the filter unit the solvent removal is started without any purposeful added waiting period, i.e. a period wherein no means are performed in order to remove the solvent. Especially it is not intended that the solvent is for instance left standing or stirred or tempered or otherwise conditioned without any additional means for solvent removal. This procedure is in contrast to standard re-crystallization steps, wherein always a certain time period is included, wherein the crystals are subjected to an Oswald ripening. Immediate in the sense of the disclosure especially intends that the removal of the solvent is initiated on a timescale of ≥10 seconds and ≤24 h, i.e. the first solvent molecules are irreversibly removed from the filtered solution in the above given timescale. In addition, it is feasible to further define that at least 1% of the solvent mass has to be removed within 30 minutes from the ibrutinib containing solvent after the filtration step. The removal of the solvent may for instance be achieved at ambient or elevated temperatures, vacuum assisted or at ambient pressure. Surprisingly, it has been found that the immediate removal of the solvent within this process yields essentially pure amorphous ibrutinib without any significant crystalline proportions. Means for the removal of the solvent are for instance evaporation of the solvent from a vessel comprising a large surface area or via a rotary evaporator. In addition to the timescale of ≥10 seconds and ≤5 h, the solvent may be removed within ≥30 seconds and ≤2.5 h, preferably ≥1 minute and ≤1.5 h and even more preferred ≥5 minutes and ≤1 h.

Another inventive aspect discloses a pharmaceutical composition comprising amorphous ibrutinib prepared according to the inventive process. The inventive amorphous ibrutinib is particularly applicable for being incorporated in pharmaceutical compositions also comprising other pharmaceutically acceptable excipients. Pharmaceutical compositions are achievable comprising good long-term stability even at high temperatures and high humidity, good processing characteristics and a favorable bioavailability.

It is also within the scope of the disclosure to disclose an oral dosage form comprising the pharmaceutical composition including the amorphous ibrutinib processed and synthesized according to the disclosure. Especially the inventive amorphous ibrutinib is suitable for being processed into oral dosage forms. This suitability can be seen in the pressure insensitivity, chemical stability and compressibility of this amorphous form.

Within a further aspect of the disclosure the oral dosage form can be a tablet. Based on the physical and chemical characteristics of the inventive amorphous ibrutinib it is found that this form is especially suited for direct compression or granulation processes resulting in tablets exhibiting an excellent stability profile and low hygroscopy. Furthermore, it has been found that this amorphous ibrutinib polymorph is compatible with a wide range of pharmaceutical excipient used for tableting.

Furthermore, the use of a pharmaceutical composition including the amorphous ibrutinib for the treatment of mantle cell lymphoma or chronic lymphocytic leukemia is within the scope of the disclosure. Within the pharmaceutical composition the amorphous ibrutinib may be at least one of the APIs (active pharmaceutical ingredient) of the composition. Furthermore, suitable pharmaceutically acceptable excipients can be present in the composition. Examples for suitable excipients include antioxidants, binders, buffering agents, bulking, agents, disintegrants, diluents, fillers, glidants, lubricants, preservatives, surfactants and/or co-surfactants.

With respect to additional advantages and features of the previously described pharmaceutical composition, the oral dosage form and the use it is explicitly referred to the disclosure of the inventive process. In addition, also aspects and features of the inventive process shall be deemed applicable and disclosed to the inventive amorphous ibrutinib and the inventive pharmaceutical composition. Furthermore, all combinations of at least two features disclosed in the claims and/or in the description are within the scope of the disclosure.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1 to 11 show

FIG. 1. a PXRD-pattern of amorphous ibrutinib precipitated from 1-propanol in a small scale experiment;

FIG. 2. a PXRD-pattern of amorphous ibrutinib precipitated from 2Me-THF in a small scale experiment;

FIG. 3. a PXRD-pattern of amorphous/crystalline ibrutinib precipitated/crystallized from 1-propanol in a medium scale experiment;

FIG. 4. a PXRD-pattern of a second experiment of amorphous/crystalline ibrutinib precipitated/crystallized from 1-propanol in a medium scale experiment;

FIG. 5. a PXRD-pattern of crystalline ibrutinib crystallized from 2Me-THF in a medium scale experiment;

FIG. 6. a PXRD-pattern of amorphous ibrutinib precipitated from benzonitrile in a small scale experiment (initial);

FIG. 7. a PXRD-pattern of amorphous ibrutinib precipitated from benzonitrile in a small scale experiment after 1 month storage time (at ambient temperature);

FIG. 8. a PXRD-pattern of amorphous ibrutinib precipitated from benzonitrile in a medium scale experiment;

FIG. 9. a PXRD-pattern of amorphous ibrutinib precipitated from methylethylketon (MEK) in a small scale experiment;

FIG. 10. a PXRD-pattern of amorphous ibrutinib precipitated from methylethylketon (MEK) in a medium scale experiment (initial);

FIG. 11. a PXRD-pattern of amorphous ibrutinib precipitated from methylethylketon (MEK) in a medium scale experiment after 1 month storage time (at ambient temperature).

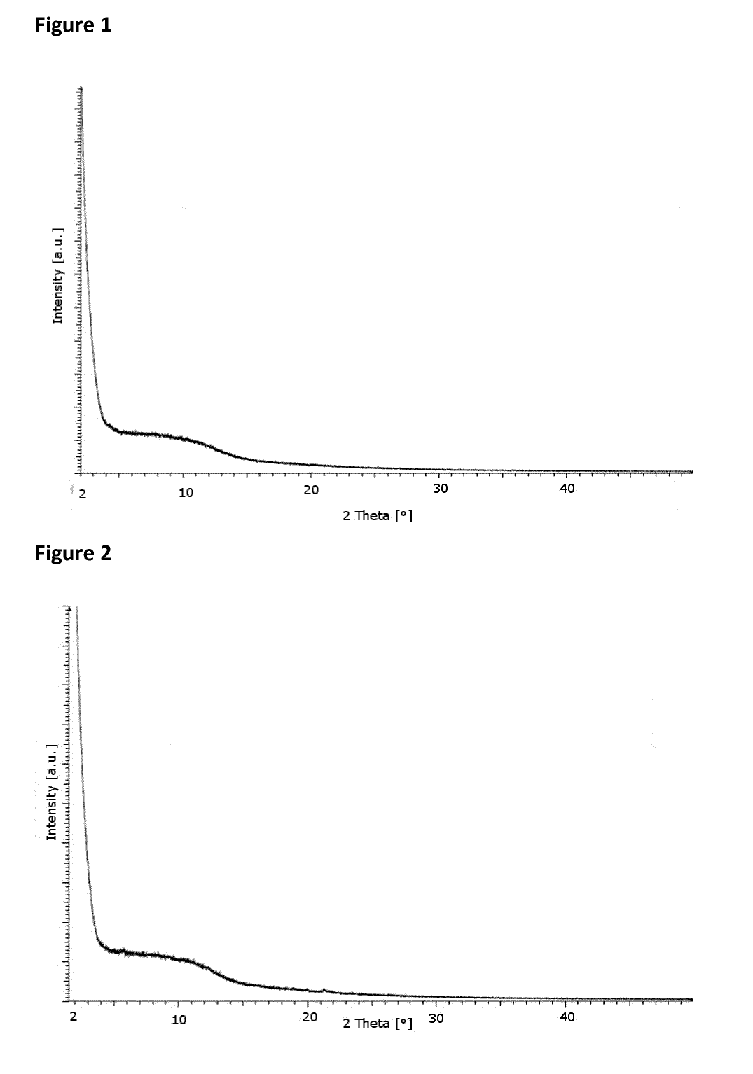

All diffraction patterns of the FIGS. 1-11 are displayed in the 2-theta range from 2° up to 50°. The PXRD-measurements were either performed in Bragg-Brentano geometry (BB) or by placing the sample in a standard glass capillary (∅=0.7 mm) and rotation of the sample. The pattern were recorded at room temperature with a D8 Bruker Advance Diffractometer (Cu-Kα1=1.54059 Å, Johansson primary beam monochromator, position sensitive detector) in transmission mode. The measurement time was 2 h.

### DETAILED DESCRIPTION

Example embodiments will now be described more fully with reference to the accompanying drawings.

FIG. 1 exhibits the diffraction pattern of ibrutinib precipitated from 1-propanol prepared in a small scale experiment. The sample is amorphous as indicated by the presence of a broad halo without any defined diffraction peaks. The ibrutinib was prepared by dissolution of 20.4 mg ibrutinib in 1.2 ml 1-propanol at 50° C., filtration of the solution (0.2 μm pore size) and evaporation of the solvent at 50° C. A colorless, glassy solid is obtained. The PXRD-pattern was recorded in BB-mode.

FIG. 2 shows the diffraction pattern of ibrutinib precipitated from 2-MeTHF prepared in a small scale experiment. The sample is amorphous as indicated by the presence of a broad halo without any defined diffraction peaks. The ibrutinib was prepared by dissolution of 19.6 mg ibrutinib in 0.5 ml 2Me-THF at 50° C., filtration of the solution (0.2 μm pore size) and evaporation of the solvent at 50° C. A colorless, glassy solid is obtained. The PXRD-pattern was recorded in BB-mode.

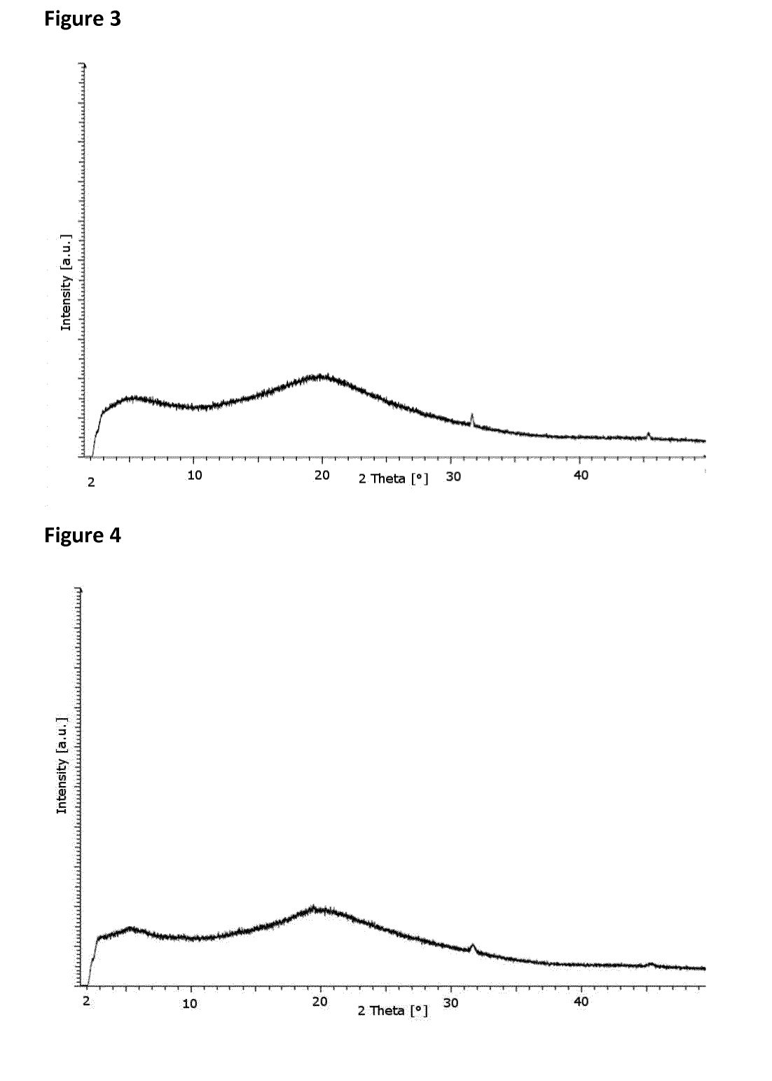

FIG. 3 displays the diffraction pattern of ibrutinib precipitated from 1-propanol prepared in an upscaling experiment. The sample is only partially amorphous as indicated by the presence of defined diffraction peaks on top of the broad halo. Peaks are for instance visible at approximately 32° and 46° indicating the presence of ordered structures in the sample. The ibrutinib was prepared by dissolution of 400 mg ibrutinib in 25 ml 1-propanol at 50° C., filtration of the solution (0.2 μm pore size) and evaporation of the solvent at 50° C. A colorless, glassy solid is obtained. The PXRD-pattern was recorded in a rotating glass capillary. A comparison of the experimental results displayed in FIG. 1 and FIG. 3 reveals that although the same drug/solvent ratio (approx. 17 mg/ml) is used the dissolved ibrutinib tends to crystallize in a defined crystalline instead of an amorphous structure. Without being bound by the theory this might be attributed to the fact that the evaporation process in the upscaling process lasts longer, favoring the formation of crystalline structures in 1-propanol.

FIG. 4 exhibits the diffraction pattern of ibrutinib precipitated from 1-propanol prepared in an upscaling experiment. The sample is only partially amorphous as indicated by the presence of defined diffraction peaks on top of the broad halo. Peaks are for instance visible at approximately 32° and 46° indicating the presence of ordered structures in the sample. The ibrutinib was prepared by dissolution of 150 mg ibrutinib in 20 ml 1-propanol at 50° C., filtration of the solution (0.2 μm pore size) and evaporation of the solvent at 50° C. A colorless, glassy solid is obtained. The PXRD-pattern was recorded in BB-mode. A comparison of the experimental results displayed in FIG. 3 and this figure reveals that although the drug/solvent ratio (approx. 7.5 mg/ml) in this experiment is much lower compared to the experiment displayed in FIG. 3 still the dissolved ibrutinib tends to crystallize in crystalline instead of amorphous structures. This experiment clearly indicates that this behavior is not caused by an insufficient dissolution of the ibrutinib in the dissolution step and remaining crystals in the solution. Such explanation is unlikely, because by using a higher solvent content the dissolution of the ibrutinib should be better compared to using lower solvent:drug-ratios. Without being bound by the theory the achievement of crystalline structures might be attributed to the fact that the evaporation process in the upscaling process lasts longer, favoring the formation of crystalline structures in 1-propanol.

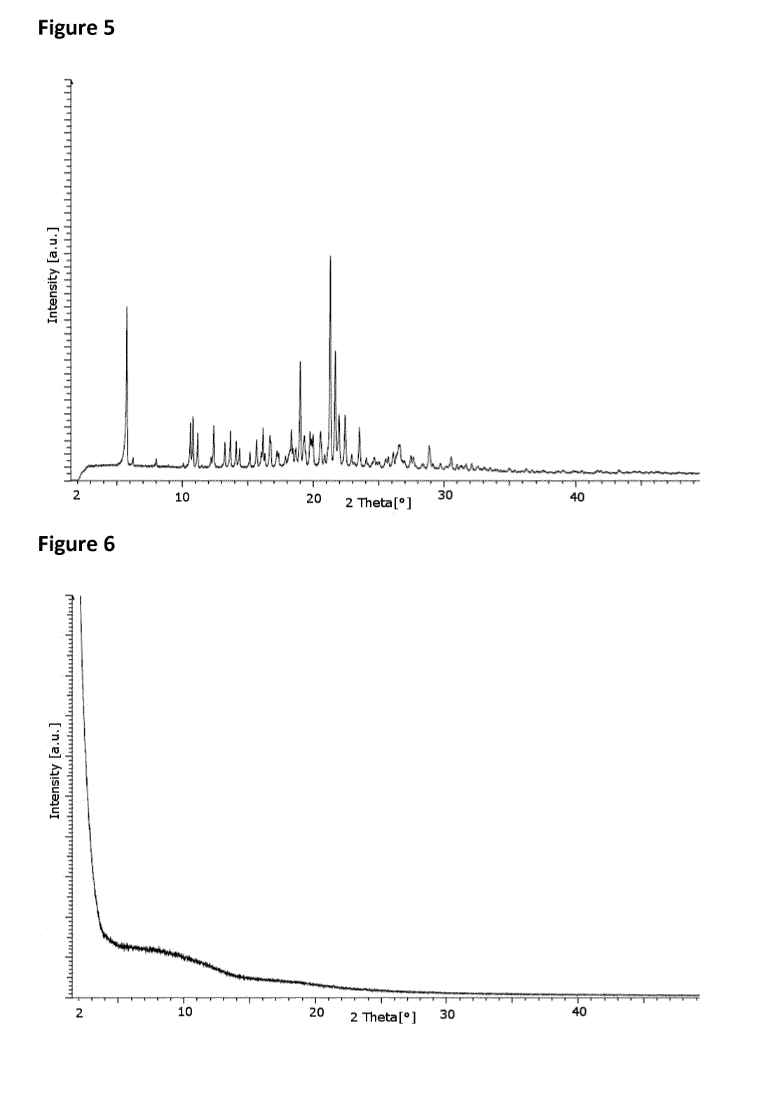

FIG. 5 displays the diffraction pattern of ibrutinib precipitated from 2-MeTHF prepared in an upscaling experiment. It can be depicted from the defined diffraction pattern that the A-form polymorph of ibrutinib is achieved. The ibrutinib was prepared by dissolution of 102 mg ibrutinib in 10 ml 2-MeTHF at 50° C., filtration of the solution (0.2 μm pore size) and evaporation of the solvent at 50° C. A colorless solid is obtained. The PXRD-pattern was recorded in a rotating glass capillary. A comparison of the experimental results displayed in FIG. 2 and FIG. 5 reveals that by using higher solvent amounts the ibrutinib tends to crystallize in a defined crystalline instead of an amorphous structure. Without being bound by the theory this might be attributed to the fact that the evaporation process in the upscaling process lasts longer, favoring the formation of crystalline structures in 2-MeTHF.

The crystallization experiments of ibrutinib performed in 1-propanol and 2-MeTHF (FIGS. 1-5) reveal that processing of the ibrutinib out of this solvent results only at a small scale in the formation of an amorphous form, whereas at larger scales only partially crystalline material is achieved.

FIG. 6 exhibits the diffraction pattern of ibrutinib precipitated from benzonitrile prepared in a small scale experiment. The sample is amorphous as indicated by the presence of a broad halo without any defined diffraction peaks. The ibrutinib was prepared by dissolution of 20.8 mg ibrutinib in 0.5 ml benzonitrile at 50° C., filtration of the solution (0.2 μm pore size) and evaporation of the solvent at 50° C. A slightly yellowish, glassy solid is obtained. The PXRD-pattern was recorded in BB-mode.

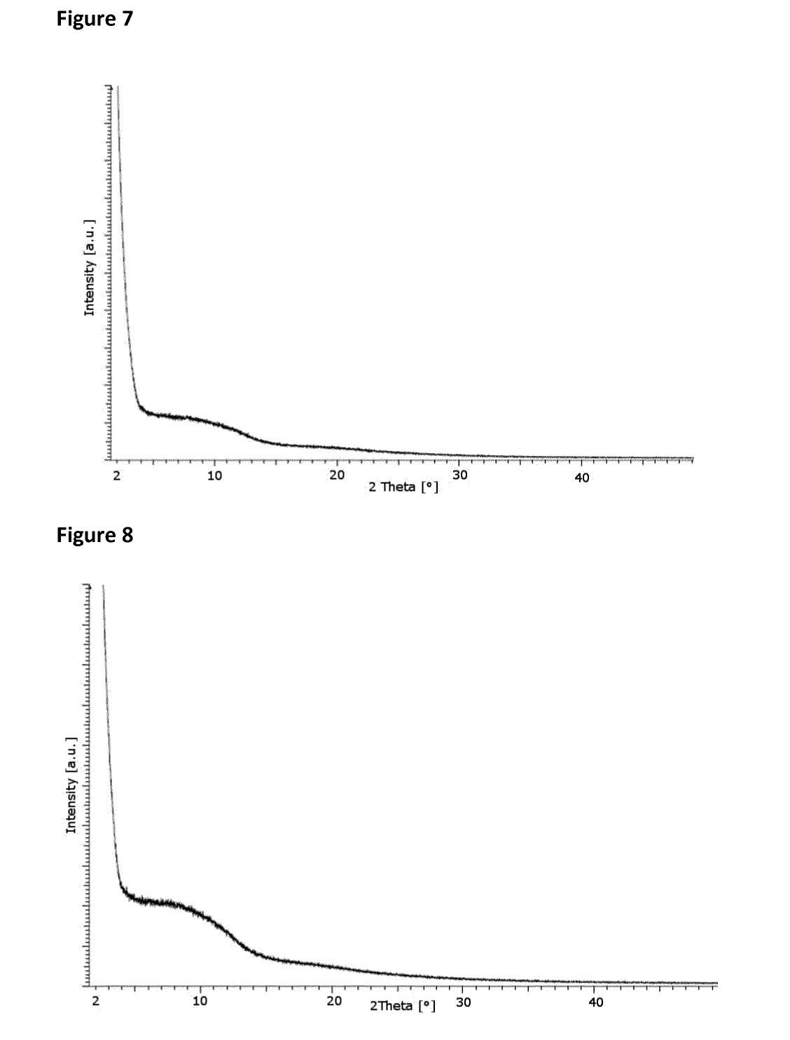

FIG. 7 shows the diffraction pattern of a stability test (1 month, ambient temperature) of the material used in FIG. 6 (small scale, benzonitrile). This pattern reveals that the amorphous form is storage stable and no crystallization of the amorphous form occurs upon storage. The PXRD-pattern was recorded in BB-mode.

FIG. 8 shows the diffraction pattern of ibrutinib precipitated from benzonitrile prepared in an upscaling experiment. The sample is amorphous as indicated by the presence of a broad halo without any defined diffraction peaks. The ibrutinib was prepared by dissolution of 101.5 mg ibrutinib in 1.5 ml benzonitrile at 50° C., filtration of the solution (0.2 μm pore size) and evaporation of the solvent at 50° C. A slightly yellowish glassy solid is obtained. The PXRD-pattern was recorded in BB-mode. A comparison of the experimental results displayed in FIG. 6 and FIG. 7 reveals that by using higher solvent amounts the ibrutinib also precipitates in an essentially amorphous structure. Without being bound by the theory this might be attributed to the special characteristics of this solvent.

As it can be deduced from the PRXD-pattern of the FIGS. 6-8 it is possible to achieve essentially amorphous ibrutinib even at larger scale experiments out of benzonitrile. This in contrast to the upscaling precipitation behavior of ibrutinib from 1-propanol and 2-MeTHF.

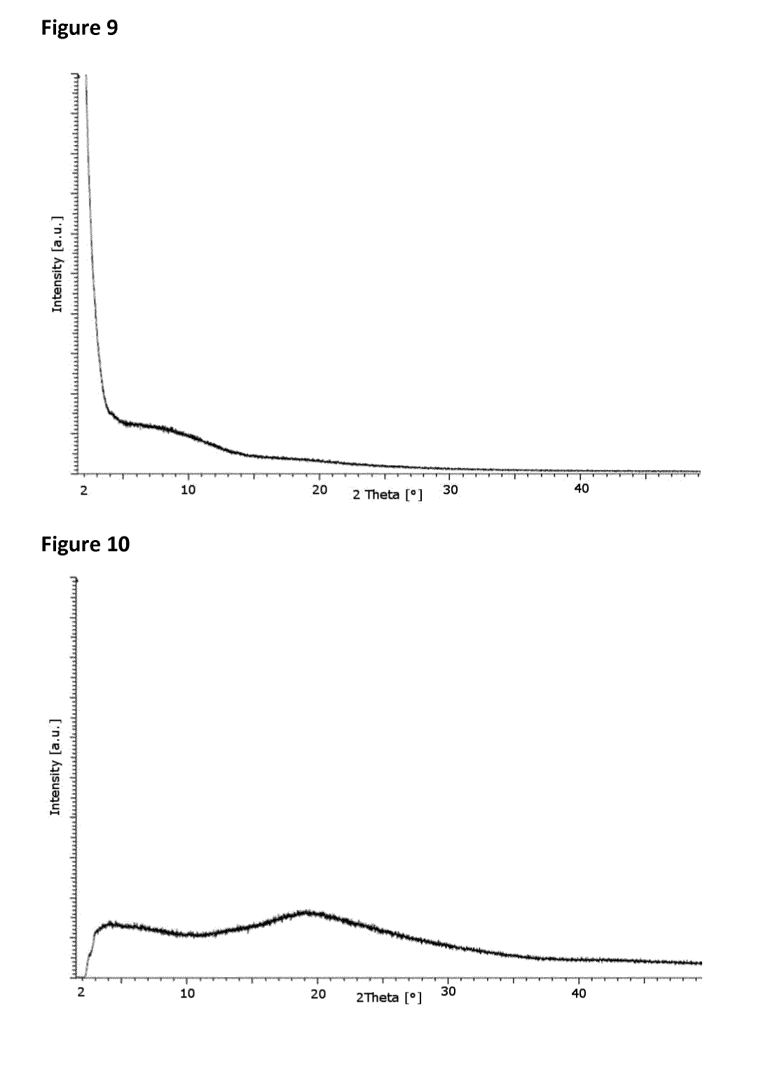

FIG. 9 exhibits the diffraction pattern of ibrutinib precipitated from methylethylketon (MEK) prepared in a small scale experiment. The sample is amorphous as indicated by the presence of a broad halo without any defined diffraction peaks. The ibrutinib was prepared by dissolution of 20.4 mg ibrutinib in 1.2 ml methylethylketon at 70° C., filtration of the solution (0.2 μm pore size) and evaporation of the solvent at 50° C. A slightly yellowish, glassy solid is obtained. The PXRD-pattern was recorded in BB-mode.

FIG. 10 shows the diffraction pattern of ibrutinib precipitated from methylethylketon prepared in an upscaling experiment. The sample is amorphous as indicated by the presence of a broad halo without any defined diffraction peaks. The ibrutinib was prepared by dissolution of 200.6 mg ibrutinib in 23 ml methylethylketon at 70° C., filtration of the solution (PTFE-KPF 0.45 μpore size) in a hot collection container under stirring and evaporation of the solvent at 70° C. A slightly yellowish, glassy solid is obtained. The PXRD-pattern was recorded in a rotating glass capillary.

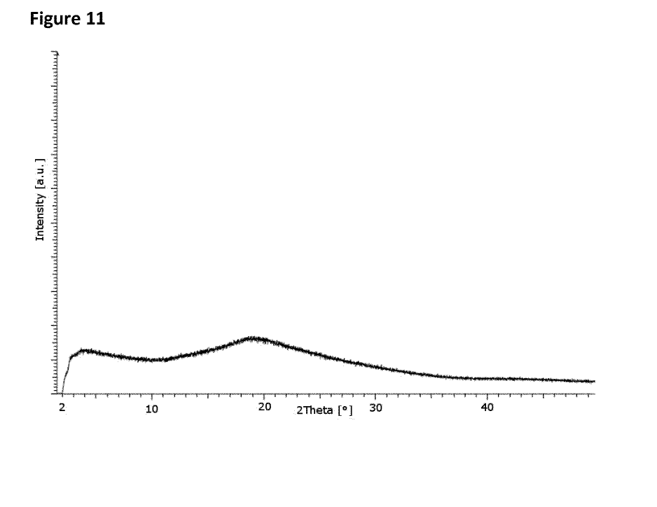

FIG. 11 displays the diffraction pattern of amorphous ibrutinib prepared as described for the material of FIG. 10 after 1 month storage at ambient temperature. The ibrutinib is still essentially amorphous as indicated by the presence of a broad halo without any defined diffraction peaks. Therefore, it is also possible to achieve storage stable, essentially amorphous ibrutinib from precipitation out of MEK.

### EXPERIMENTAL EXAMPLES

### Example 1

The compounds described herein were synthesized according to the following steps outlined in the Scheme 1. A detailed illustrative example for the synthesis of compound (4), i.e. 1-((R)-3-(4-amino-3-(4-phenoxyphenyl)pyrazolo[3,4-d]pyrimidin-1-yl]-1-piperidyl]prop-2-en-1-one) is depicted in the following

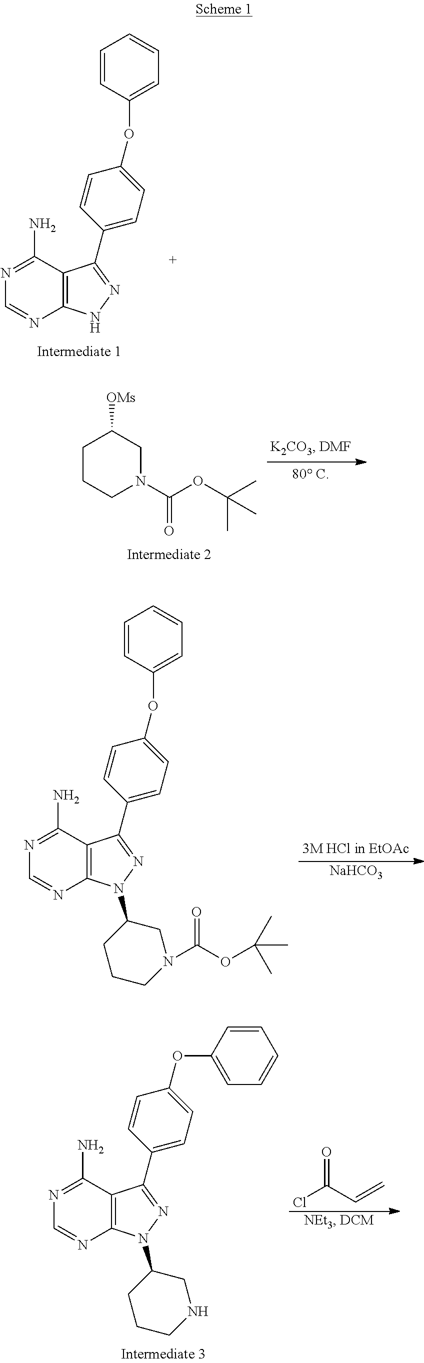

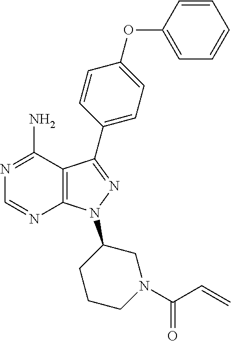 [SMILES: C=CC(=O)N1CCC[C@@H](n2nc(-c3ccc(Oc4ccccc4)cc3)c3c(N)ncnc32)C1]

Intermediate 1 (5 g) was dissolved in dry DMF (50 mL) and potassium carbonate (8.8 g) was added. The suspension was stirred at ambient temperature for 2 h. After dropwise addition of Intermediate 2 (9 g) dissolved in DMF (10 mL) the reaction mixture was heated at 80° C. for 14 h. The organic layer was separated and the water layer extracted with EtOAc (3×20 mL). The organic layers were combined and dried over Na₂SO₄. Solvent evaporation at reduced pressure and recrystallization result in 6.3 g (80%) tert-butyl-(3R)-3-[4-amino-3-(4-phenoxyphenyl)pyrazolo[3,4-d]pyrimidin-1-yl]piperidine -1-carboxylate.

Tert-butyl-(1R)-3-4-amino-3-(4-phenoxyphenyl)pyrazolo[3,4-d]pyrimidine-1-carboxylate (3 g) was dissolved in 3 M HCl-EtOAc (15 mL). After 30 min the solution was treated with saturated sodium bicarbonate solution. The organic layer was separated and the water layer extracted with DCM (3×10 mL). The organic layers were combined and dried over Na₂SO₄. Evaporation of the solvent gives 2.1 g (91%) 3-(4-phenoxyphenyl)-1-[(3R)-3-piperidyl]pyrazolo[3,4-d]pyrimidin-4-amine.

3-(4-Phenoxyphenyl)-1-[(3R)-3-piperidyl]pyrazolo[3,4-d]pyrimidin-4-amine (0.5 g) was dissolved in DCM (25 mL) and NEt₃ (0.47 mL) was added, following dropwise addition of acryloyl chloride (0.104 mL) dissolved in DCM (5 mL). The reaction mixture was washed with 1N citric acid solution and then with brine. The organic layer was dried with Na₂SO₄. A solvent exchange was performed with benzonitrile. After filtration of the solution (0.2 μm pore size) and evaporation of the solvent at 50° C. 1-[(3R)-3-[4-amino-3-(4-phenoxyphenyl)pyrazolo[3,4-d]pyrimidin-1-yl]-1-piperidyl]prop-2-en-1-one was obtained.

### Educt Preparation (Intermediate 1)

### Synthesis of 4-Amino-3-(4-phenoxyphenyl)-1H-pyrazolo[3,4-d]pyrimidine

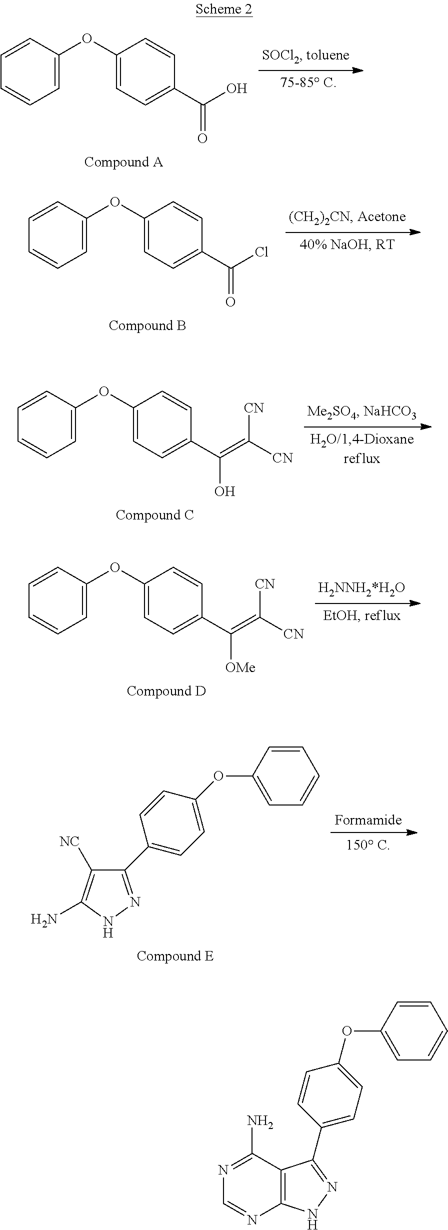 [SMILES: O=C(O)c1ccc(Oc2ccccc2)cc1 ; O=C(Cl)c1ccc(Oc2ccccc2)cc1 ; N#CC(C#N)=C(O)c1ccc(Oc2ccccc2)cc1 ; COC(=C(C#N)C#N)c1ccc(Oc2ccccc2)cc1 ; N#Cc1c(-c2ccc(Oc3ccccc3)cc2)n[nH]c1N ; Nc1ncnc2[nH]nc(-c3ccc(Oc4ccccc4)cc3)c12]

4-Phenoxybenzoic acid (10 g) was suspended in toluene (30 ml) and after that thionyl chloride (7 mL) was added and the suspension stirred at 75-85° C. Thionyl chloride and toluene was removed by distillation (resulting in compound B).

The resulting acid chloride was dissolved in acetone (40 mL), malononitrile (3.3 g) was added and the solution was stirred at 10° C. Aqueous sodium hydroxide solution (40%) was added dropwise to this solution under vigorous stirring, keeping the temperature constant below 30° C. After that the reaction mixture was stirred for further 2 h at room temperature. After the reaction was completed the reaction mixture was poured into water and 10-12% hydrochloric acid was added in order to adjust the pH <1.5. A precipitate formed. The precipitate was filtered off, washed with water and dried under reduced pressure. After drying 11.3 g (90%) of 1,1-dicyano-2-hydroxy-2-(4-phenoxyphenyl)ethen (compound C) were obtained.

1,1-Dicyano-2-hydroxy-2-(4-phenoxyphenyl)ethene (11 g) and NaHCO₃ (28 g) were suspended in a mixture of 1,4-Dioxane (70 mL) and H₂O (11 mL). Dimethylsulfate (29 mL) was added dropwise under vigorous stirring. The mixture was refluxed for 2 h and treated with cold water. The mixture was extracted with EtOAc (3×30 mL) and the combined extracts were dried over Na₂SO₄. Evaporation of the solvent under reduced pressure gives 11.5 g crude product as dark brown oil. After the treatment of the crude product with cold ethanol at 5-10° C. 6.5 g (57%) of 1,1-dicyano-2-methoxy-2-(4-phenoxyphenyl)ethen were obtained (compound D).

1,1-Dicyano-2-methoxy-2-(4-phenoxyphenyl)ethen (6 g) was suspended in ethanol (25 mL) and hydrazine hydrate (6 g) was added. After 1 h of reflux the forming precipitate was filtered and washed with ethanol/water (¼ mixture). 6 g (92%) of 3-amino-4-cyano-5-(4-phenoxyphenyl)pyrazole were obtained.

3-Amino-4-cyano-5-(4-phenoxyphenyl)pyrazole (6 g) was dissolved in formamide (60 mL) and stirred at 150° C. for 8 h. The reaction mixture was cooled to room temperature, water (60 mL) was added and the forming precipitate collected. The precipitate was washed with methanol/water (30 mL, ⅕ mixture) to give 6 g (90%) of 4-amino-3-(4-phenoxyphenyl)-1H-pyrazolo[3,4-d]pyrimidine (Intermediate 1).

### Educt preparation (Intermediate 2)

### Synthesis of tert-butyl-3S-(methylsulfonyloxy) piperidine-1-carboxylate

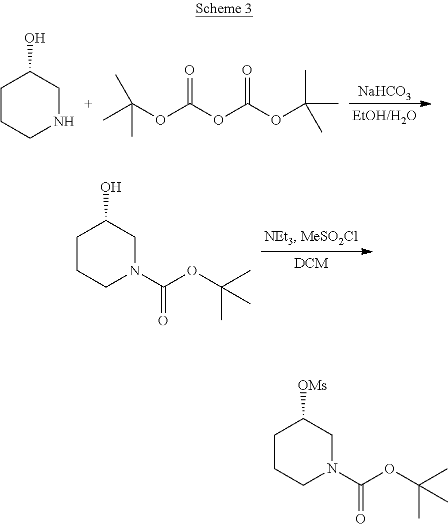

1-N-(3S)-hydroxypiperidine hydrochloride (10 g) was dissolved in H₂O/EtOH (480 mL, 1:1, v/v). Then, NaHCO₃ (60 g) was added followed by stirring at room temperature and the addition of Boc₂O (25 g). The reaction was completed in 4 h. The mixture was filtered and the filtrate was evaporated. The crude product was then dissolved in dry DCM (120 mL) and filtered from sodium bicarbonate.

To the filtrate NEt₃ (16 mL) was added followed by dropwise addition of methanesulfonyl chloride (5 mL) at 0° C. The reaction mixture was stirred for 3 h at room temperature. After the reaction was completed the reaction mixture was diluted with 0.5 M HCl solution. The organic layer was separated and the water layer extracted with DCM (3×10 mL). The organic layers were combined and dried over Na₂SO₄. The evaporation of the solvent under reduced pressure gives 16 g (90%) tert-butyl-3S-(methylsulfonyloxy)piperidine-1-carboxylate as solid (Intermediate 2).

### Example 2

In the fourth example Ibrutinib is synthesized according to the steps outlined in the following Scheme 4:

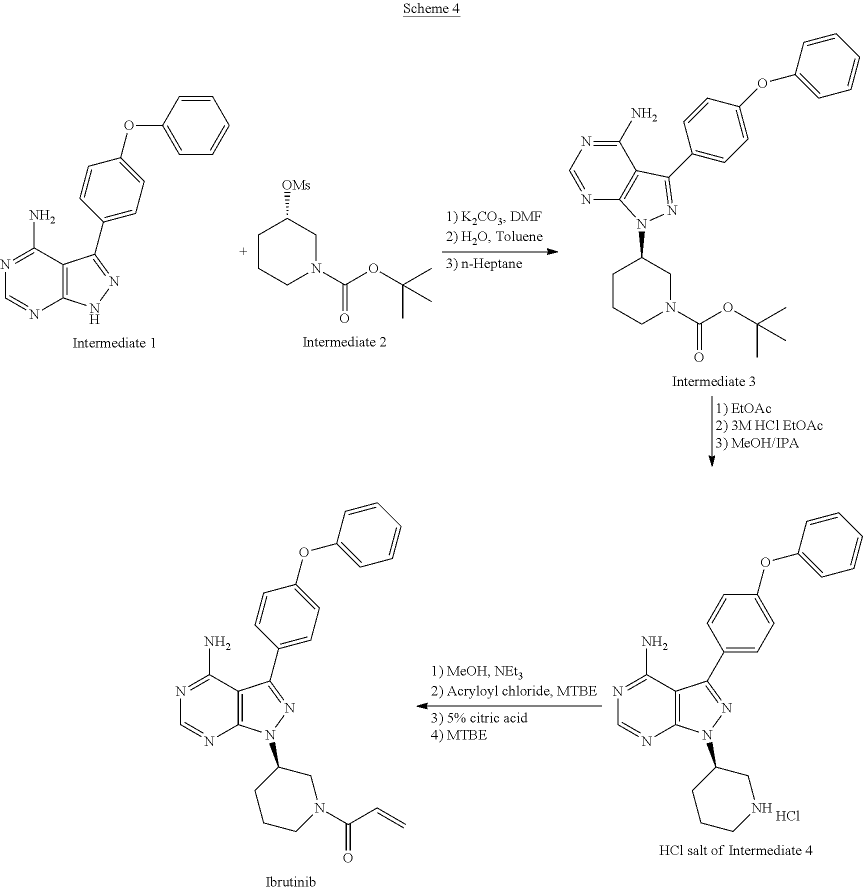 [SMILES: Nc1ncnc2[nH]nc(-c3ccc(Oc4ccccc4)cc3)c12 ; CC(C)(C)OC(=O)N1CCC[C@H](OS(C)(=O)=O)C1 ; CC(C)(C)OC(=O)N1CCC[C@@H](n2nc(-c3ccc(Oc4ccccc4)cc3)c3c(N)ncnc32)C1 ; C=CC(=O)N1CCC[C@@H](n2nc(-c3ccc(Oc4ccccc4)cc3)c3c(N)ncnc32)C1 ; Nc1ncnc2c1c(-c1ccc(Oc3ccccc3)cc1)nn2[C@@H]1CCCNC1 ; Cl]

### Preparation of intermediate 3

25 g (0.082.4 mol, 1 eq.) of Intermediate 1 and 45.56 g (0.329 mol, 4 eq.) K₂CO₃ were slurried in 400 ml DMF. The suspension was stirred at ambient temperature for 10 minutes. After 46.05 g (0.164 mol, 2 eq.) of intermediate 2 dissolved in 100 ml DMF was added dropwise to the reaction mixture. The reaction mixture was heated to 80° C. and stirred. Afterwards, the reaction mixture cooled to ambient temperature. 1000 ml water and 250 ml toluene were added to the reaction mixture, resulting in the formation of two phases. The phases were separated and the aqueous phase was extracted with 200 ml toluene. The organic phases were combined and washed with 300 ml 20% brine solution. The solution was dried over 30 g Na₂SO₄. The organic phase was filtrated and the solvent was evaporated. After purification with 100 ml n-heptane the crystals were filtered and dried at 60° C. under vacuum (180 mbar) to yield 31.49 g (0.0647 mol; 78.5%) intermediate 3. The melting point was 144-146° C.

### Preparation of the HCl-salt of intermediate 4

50 g (0.1027 mol) of crude intermediate 3 was suspended in 100 ml ethyl acetate at ambient temperature. 200 ml M HCl in EtOAc solution were added under stirring to this suspension. After 15 min the formation of a bulky mass was observed. The stirring was continued at ambient temperature overnight. The resulting precipitate was filtered, washed with 50 ml ethyl acetate and dried at 80° C. under vacuum (180 mbar), yielding 43.35 g (99.3%)

After purification from a MeOH/iPrOH mixture 26 g (0.0613 mol, 83%) of the HCl salt of intermediate 4 was obtained in the form of a milky powder (melting point 264-266° C.).

### Preparation of Ibrutinib

2.45 g (5.78 mmol, 1 eq.) HCl salt of intermediate 4 was dissolved in 50 ml dry methanol at ambient temperature and 2.02 g (20 mmol, 3.5 eq.) of triethylamine was added. The mixture was cooled to −12° C. and 0.628 g (6.94 mmol, 1.2 eq.) acryloyl chloride dissolved in 20 ml methyl tert.-butyl ether was added dropwise into the light yellow clear solution. The temperature of reaction mixture was kept between −12° C. to −10° C. 50 ml methyl tert.-butyl ether and 50 ml 5% citric acid solution were added to the reaction mixture. Two separate layers formed. The organic phase was isolated and the aqueous phase was extracted with 2×30ml methyl tert.-butyl ether. The organic phases were combined and washed with 50 ml saturated sodium carbonate solution followed by 40 ml saturated brine solution. The organic phase was evaporated at room temperature and 2.2 g (4.99 mol, 86.2%) white solid were obtained

### Preparation of Intermediate 2

Intermediate 2 was synthesized following a two step procedure.

In a first step a compound H was synthesized according to the following scheme:

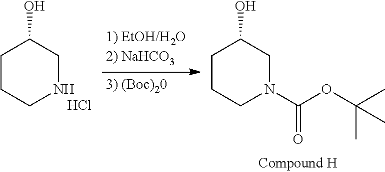

50 g (0.3633 mol, 1 eq.) (S)-3-Hydroxypiperidine hydrochloride was dissolved in 200 ml ethanol and 200 ml water (1:1) mixture and 243 g (2.9064 mol, 8 eq.) sodium bicarbonate was added to the solution at room temperature (21-22° C.). 95.13 g (0.4359 mol, 1.2 eq.) di-tert-butyl dicarbonate was dissolved in 100 ml ethanol and the mixture is added in portions. The reaction mixture was stirred overnight. Afterwards, the reaction mixture was filtered off, washed in ethanol and the filtrate was evaporated. A yellowish oil (product) and a white precipitate (sodium bicarbonate) were obtained as residues. The residues were dissolved in 50 ml dichloromethane, filtered from sodium bicarbonate and the filtrate again was evaporated. A colourless oil was obtained as residue, which was at room temperature stepwise precipitated to give white crystals (yield 72.25 g, 0.3589 mol, 98.9%, melting point 49-51° C.).

In a second step Compound H is converted to the Intermediate 2 according to the following scheme:

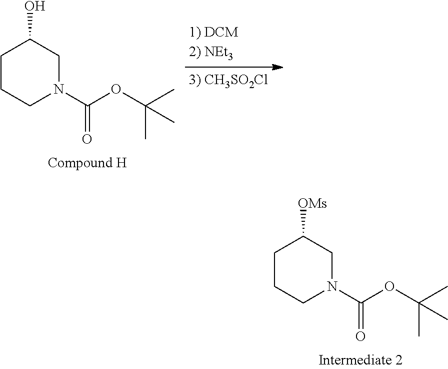 [SMILES: CC(C)(C)OC(=O)N1CCC[C@H](O)C1 ; CC(C)(C)OC(=O)N1CCC[C@H](OS(C)(=O)=O)C1]

129.19 g (0.6419 mol, 1 eq.) crude tert-butyl (3S)-3-hydroxypiperidine-1-carboxylate was dissolved in 800 ml dichloromethane. 142.88 g (1.412 mol, 2.2 eq.) trimethylamine was added to the solution at room temperature (RT=21-22° C.). The reaction mixture was cooled to −5° C. and 95.5 g (0.834 mol, 1.3 eq.) methansulfonyl chloride was added dropwise to the reaction mixture. The ice bath was removed and the mixture stirred overnight at room temperature. 120 ml 1N HCl solution were added to the reaction mixture to adjust the pH to 3-4. Two layers formed. The organic phase was isolated and the water phase extracted with 2×50 ml dichloromethane. The organic phases were combined and the solvent evaporated. Light yellow crystals were obtained. The crystals were suspended in 700 ml water and stirred for 4 h at room temperature. The crystals were filtered and dried under vacuum (yield 165.85 g, 0.5938 mol, melting point 90-91° C.).

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are to be regarded as a departure from the disclosure, and all such modifications are intended to be included within the scope of the disclosure.

1. A process for the production of amorphous ibrutinib at least comprising the following synthesis steps:
a) reacting compound (1) (3-(4-phenoxyphenyl)-1H-pyrazolo[3,4-d]pyrimidin-4-amine) and an in 3 position functionalized and amino-protected piperidine, wherein the 3 position is functionalized by a leaving group X and the piperidine amino-group is protected by the amino-protecting group Z, to yield compound (2) (1-[(3R)-3-[4-Amino-3-(4-phenoxyphenyl)pyrazolo[3,4-d]pyrimidin-1-yl]piperidin-1-yl]Z) 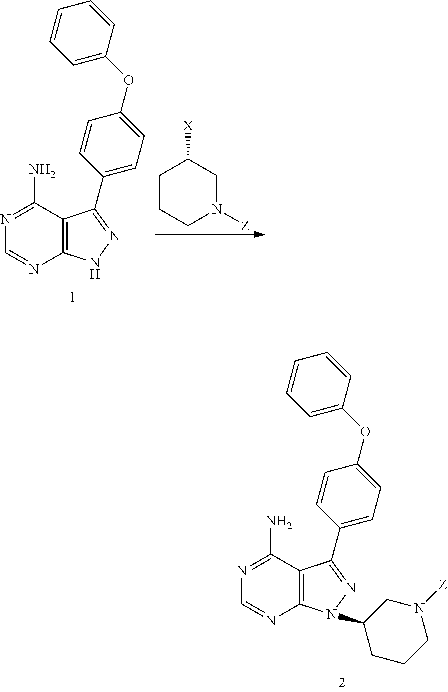
b) deprotection of compound (2) to yield compound (3) (1-[(3R)-3-[4-Amino-3-(4-phenoxyphenyl)pyrazolo[3,4-d]pyrimidin-1-yl]piperidin]); 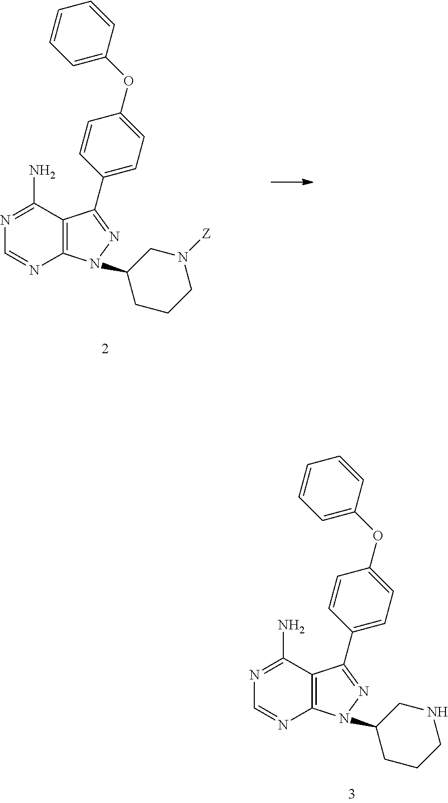
c) reacting compound (3) and acryloyl chloride (2-propenoyl chloride) in an pharmaceutically acceptable solvent to yield compound (4) ibrutinib (1-[(3R)-3-[4-Amino-3-(4-phenoxyphenyl)pyrazolo[3,4-d]pyrimidin-1-yl]piperidin-1-yl]prop-2-en-1-one); 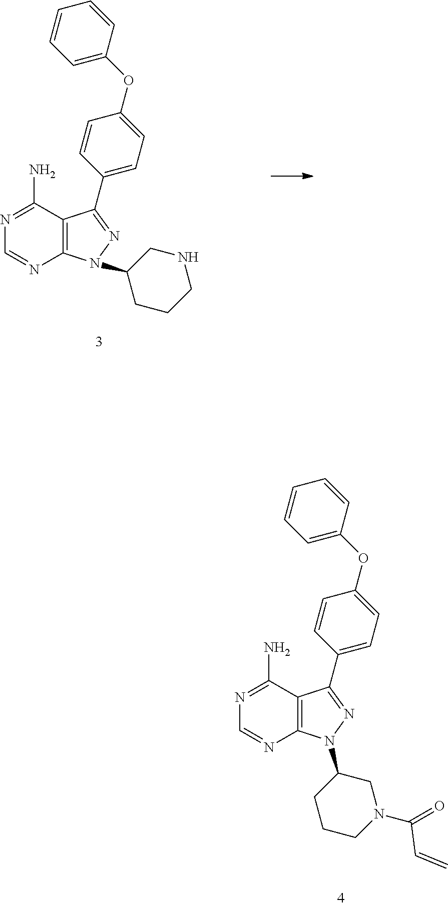 [SMILES: Nc1ncnc2c1c(-c1ccc(Oc3ccccc3)cc1)nn2[C@@H]1CCCNC1 ; C=CC(=O)N1CCC[C@@H](n2nc(-c3ccc(Oc4ccccc4)cc3)c3c(N)ncnc32)C1]
d) filtration of the reaction mixture obtained in step c); and
e) evaporation of the solvent to precipitate essentially amorphous compound (4); wherein the pharmaceutically acceptable solvent of step c) comprises an electric dipole moment μ/D of ≥2 and ≤8.

2. The process according to claim 1, wherein the pharmaceutically acceptable solvent of step c) comprises an electric dipole moment μ/D of ≥2.5 and ≤5.

3. The process according to claim 1, wherein the pharmaceutically acceptable solvent of step c) comprises an electric dipole moment μ/D of ≥2.5 and ≤5 and a boiling point ≥75° C. and ≤200° C.

4. The process according claim 1, wherein the solvent in step c) is selected from the group consisting of MEK, benzonitrile or mixtures thereof.

5. The process according to claim 1, wherein the leaving group X of the piperidine in step a) is selected from the group consisting of halogenide, mesylate, triflate, tosylate, benzenesulfonate.

6. The process according to claim 1, wherein the amino-protecting group Z of the piperidine in step a) is selected from the group consisting of Boc, Cbz, Tosyl, Mesyl, Triflat, Benzyl, Fmoc, substituted or unsubstituted Acetyl, Benzoyl, Tolyl.

7. The process according to claim 1, wherein the deprotection step b) is performed acid or metal catalyzed.

8. The process according to claim 1, wherein the filtration step d) comprises passing the reaction mixture of step c) through a filter medium comprising pores sizes in the range of ≥0.001 μm and ≤5 μm.

9. The process according to claim 1, wherein the filtration step d) is performed in a temperature range of ≥25° C. and ≤100° C.

10. The process according to claim 1, wherein the evaporation step e) is performed at a pressure of ≥900 hPa and ≤1200 hPa.

11. The process according to claim 1, wherein the evaporation step e) is started immediately after the filtering step.

12. A pharmaceutical composition comprising amorphous ibrutinib prepared by a process according to claim 1.

13. An oral dosage form comprising the pharmaceutical composition according to claim 12.

14. The oral dosage form according to claim 13, wherein the dosage form is a tablet.

15. The use of the pharmaceutical composition according to claim 12 for the treatment of mantle cell lymphoma or chronic lymphocytic leukemia.
